(19) United States
(12) Patent Application Publication
(10) Pub. No.: US 2016/0281280 A1
(43) Pub. Date: Sep. 29, 2016

(54) FIBER STRUCTURE, AND BIOCHIP, SUBSTRATE FOR CELL CULTURE AND FILTER, EACH OF WHICH USES SAID FIBER STRUCTURE

(71) Applicant: PANASONIC INTELLECTUAL PROPERTY MANAGEMENT CO., LTD., Osaka-shi (JP)

(72) Inventors: ATSUSHI SHUNORI, Osaka (JP); MAKOTO TAKAHASHI, Tokushima (JP); TAKUYA OKA, Kyoto (JP)

(21) Appl. No.: 14/778,570

(22) PCT Filed: Mar. 17, 2014
(86) PCT No.: PCT/JP2014/001500

§ 371 Date: Sep. 18, 2015

(30) Foreign Application Priority Data
Mar. 25, 2013 (JP) 2013-061411

(51) Int. Cl. D04H 1/4391; B32B 5/12; B01D 39/20; B32B 15/14; B32B 27/12; D04H 13/00; C12M 1/12; B32B 9/00

(52) U.S. Cl. CPC D04H 1/4391; C12M 25/02; B32B 5/12; B32B 9/005; B32B 15/14; B32B 27/12; D04H 13/00; B01D 39/20; B32B 2262/10; B32B 2307/702

(57) ABSTRACT

A fibrous structure includes a sheet-like structure and a columnar structure. The sheet-like structure includes first fibers intertwining with each other and first pores formed among the fibers. The columnar structure includes columnar bodies each made of a second fiber and oriented in the first direction and second pores formed between the columnar bodies. The columnar bodies are each in contact, at a first end thereof, with the sheet-like structure.

### TECHNICAL FIELD

The present technical field relates to a fibrous structure used for various electronic devices and cell culture, and also relates to a biochip, a cell culture substrate, and a filter each of which includes the fibrous structure.

BACKGROUND ART

Conventional fibrous structures are made of inorganic materials, such as silicon dioxide and glass, or organic materials, such as nitrocellulose, nylon, polyvinylidene difluoride, polyester, and acrylic. These structures are used for biochips, cell culture substrates, filters, etc.

In the case of using such a fibrous structure in a biochip, a known technique is to adsorb probes, which selectively bond to detection targets contained in an aqueous solution, on the surface of the reaction field (sensor region) of the biochip. The probes and the targets interact with each other, and the strength of the interaction is detected by fluorescence or other means. The use of the fibrous structure as the reaction field increases the surface area of the biochip, thereby improving the interaction between the probes and the targets.

The fibrous structure can also be used as a scaffold on which cells are cultured. It is preferable to use the scaffold (cell culture substrate) to which cells are adsorbed because it improves the efficiency of cell culture. The scaffold also promotes the elimination of waste products from the cells, thereby further improving the efficiency of cell culture.

Conventional techniques related to the present application are shown, for example, in Patent Literatures 1 and 2.

### CITATION LIST

### Patent Literatures

PTL 1: Japanese Unexamined Patent Application Publication No. 2007-44835

PTL2: WO2011/135801

### SUMMARY OF THE INVENTION

A fibrous structure includes a sheet-like structure and a columnar structure. The sheet-like structure includes first fibers intertwining with each other and first pores formed among the fibers. The columnar structure includes columnar bodies each made of a second fiber and oriented in the first direction and second pores formed between the columnar bodies. The columnar bodies are each in contact, at a first end thereof, with the sheet-like structure.

### BRIEF DESCRIPTION OF DRAWINGS

FIG. 1 is a conceptual view of a fibrous structure in accordance with a first exemplary embodiment.

FIG. 2A is a cross-sectional SEM image of the fibrous structure in accordance with the first exemplary embodiment.

FIG. 2B is a sectional view taken along line 2B-2B of FIG. 2A.

FIG. 2C is a sectional view taken along line 2C-2C of FIG. 2A.

FIG. 3 is an SEM image of a sheet-like structure contained in the fibrous structure in accordance with the first exemplary embodiment.

FIG. 4A is a side view showing a step of a method of manufacturing the fibrous structure in accordance with the first exemplary embodiment.

FIG. 4B is a side view showing a step of the method of manufacturing the fibrous structure in accordance with the first exemplary embodiment.

FIG. 4C is a side view showing a step of the method of manufacturing the fibrous structure in accordance with the first exemplary embodiment.

FIG. 4D is a side view showing a step of the method of manufacturing the fibrous structure in accordance with the first exemplary embodiment.

FIG. 4E is a side view showing a step of the method of manufacturing the fibrous structure in accordance with the first exemplary embodiment.

FIG. 4F is a side view showing a step of the method of manufacturing the fibrous structure in accordance with the first exemplary embodiment.

FIG. 5 is a conceptual view of a biochip in accordance with a second exemplary embodiment, which includes the fibrous structure.

FIG. 6 is an enlarged view of an essential part of FIG. 5.

FIG. 7 is a conceptual view of a cell culture substrate in accordance with a third exemplary embodiment, which includes the fibrous structure.

FIG. 8 is a conceptual view of a filter in accordance with a fourth exemplary embodiment, which includes the fibrous structure.

### DESCRIPTION OF EMBODIMENTS

Conventional problems will now be described prior to describing exemplary embodiments. In the case of using a fibrous structure in a biochip, if the structure does not include enough pores, nonspecifically adsorbed substances may not be washed off thoroughly. The substances remaining in the fibrous structure would increase background noise during detection, thereby decreasing the detection sensitivity. In the case of using a fibrous structure in a cell culture substrate, on the other hand, if the structure does not include enough pores, the fibrous structure does not have a high permeability for waste products (liquid permeability), possibly inhibiting effective cell culture. Thus, it has been expected to develop fibrous structures with improved properties, such as larger surface area, higher porosity, and higher permeability.

The following is a description of the fibrous structure in accordance with exemplary embodiments, which have been developed to solve the above-described conventional problems.

### First Exemplary Embodiment

Fibrous structure 1 in accordance with a first exemplary embodiment will now be described with reference to drawings.

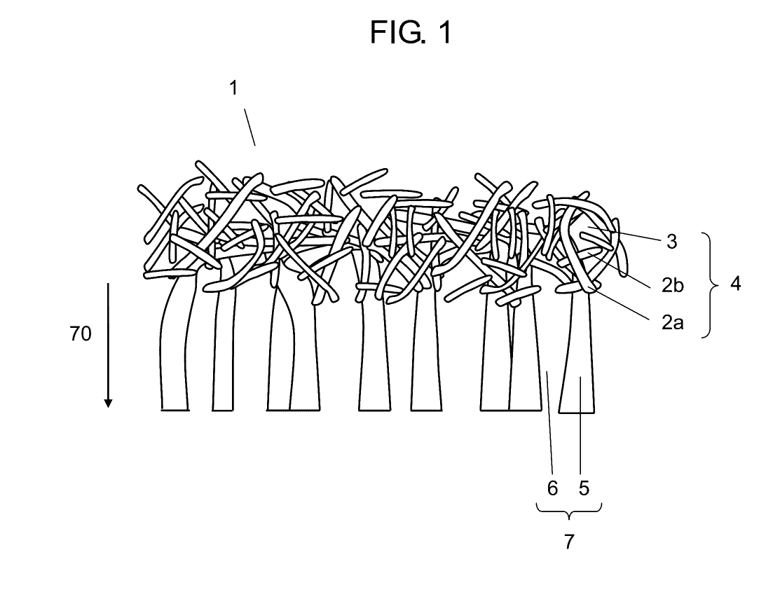

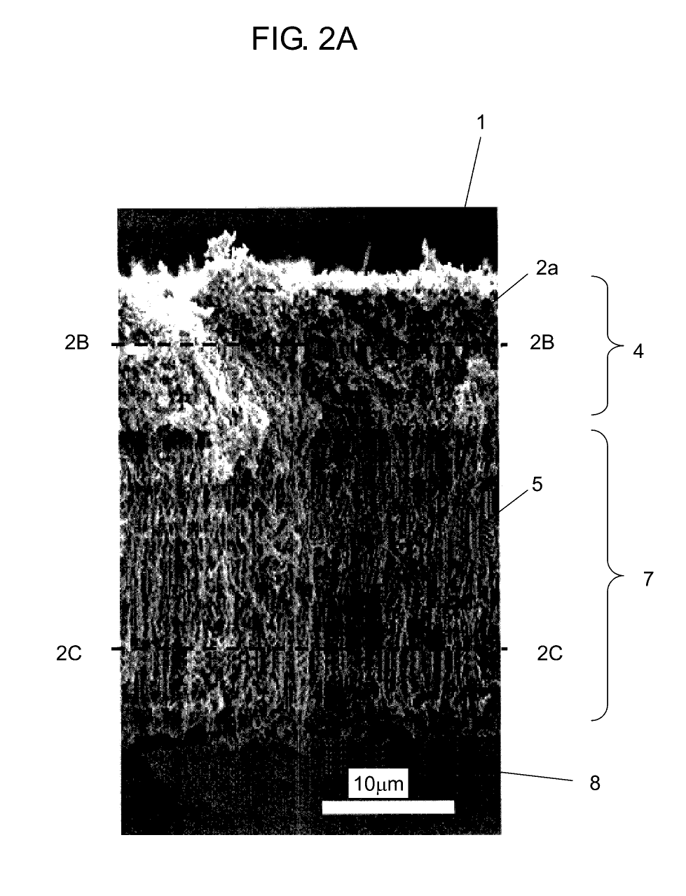

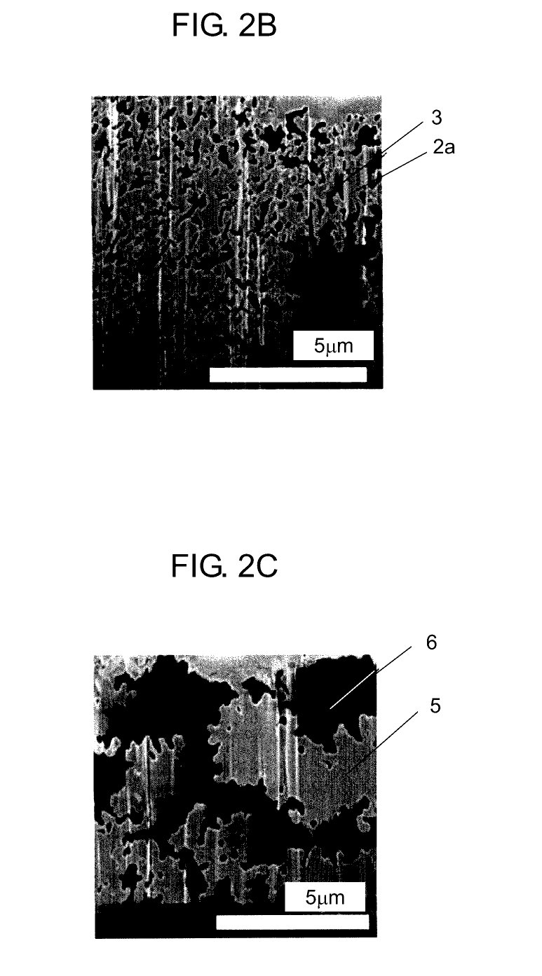

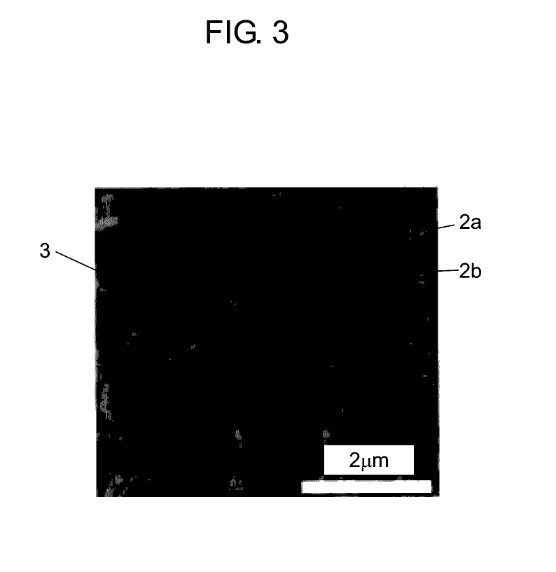

FIG. 1 is a conceptual view of fibrous structure 1 in accordance with the first exemplary embodiment. FIG. 2A is a cross-sectional SEM image of fibrous structure 1. FIG. 2B is a sectional view taken along line 2B-2B of FIG. 2A. FIG. 2C is a sectional view taken along line 2C-2C of FIG. 2A. FIG. 3 is an SEM image of sheet-like structure 4 contained in fibrous structure 1.

Fibrous structure 1 includes sheet-like structure 4 and columnar structure 7. Sheet-like structure 4 includes fibers 2a (first fibers) intertwining with each other and first pores 3 formed among fibers 2a. Columnar structure 7 includes columnar bodies 5 each made of a second fiber and oriented in first direction 70 and second pores 6 formed between columnar bodies 5. Columnar bodies 5 are each in contact, at a first end thereof, with sheet-like structure 4.

Sheet-like structure 4 is hardly dispersed when soaked in a solution because fibers 2a intertwine with each other. Fibers 2b are preferably embedded in first pores 3 formed by the intertwining of fibers 2a. This allows sheet-like structure 4 to be highly dense.

More specifically, fibers 2a have thicknesses of 0.01 to 1 μm and lengths of 0.2 to 20 μm and slightly bend to intertwine with each other. Fibers 2b have thicknesses of 0.01 to 1 μm and lengths of 0.05 to 5 μm. The average length of fibers 2a is larger than that of fibers 2b. These configurations allow fibers 2b to become embedded in first pores 3. It is further preferable that fibers 2a have an aspect ratio of 20 or more, and that fibers 2b have an aspect ratio of 1 or more and less than 5. Limiting the aspect ratios to these ranges makes the sheet-like structure much denser.

Columnar bodies 5 are in contact with sheet-like structure 4 and oriented in a direction (first direction 70) substantially perpendicular to structure 4.

Columnar bodies 5 have a larger average thickness than fibers 2a and 2b. They may be a bundle of intertwined fibers 2a and 2b instead of being a single fiber.

Since columnar bodies 5 are oriented in the direction substantially perpendicular to sheet-like structure 4, columnar structure 7 is hardly deformed under load in that direction. Therefore, fibrous structure 1 can be observed with a laser scanner or a microscope without deviation of the focal length, thereby being useful in biochips and cell culture substrates.

Second pores 6 have a larger volume than first pores 3. In the case of using fibrous structure 1 in a biochip, this feature provides liquid permeability high enough to facilitate the discharge of a cleaning solution, thereby reducing nonspecific adsorption, and then, background noise during detection. In the case of using fibrous structure 1 in a cell culture substrate, this feature facilitates the discharge of waste products.

Fibers 2a, 2b and columnar bodies 5 are preferably made of silicon dioxide (SiO₂). They can be easily surface-treated because silicon dioxide is highly biocompatible and chemically and thermally stable.

Silicon dioxide also has high chemical resistance, so that fibers 2a, 2b and columnar bodies 5 are hardly damaged by chemical treatment.

Silicon dioxide further has heat resistance higher than 1000° C., so that fibers 2a, 2b and columnar bodies 5 are resistant to the heat applied during surface treatment or sterilization.

Silicon dioxide further has low autofluorescence. When silicon dioxide is used for a biochip, and fluorescence is used for detection, the background noise is low and the detection sensitivity is high. Meanwhile, when silicon dioxide is used for a cell culture substrate, noise is low during the fluorescence observation of cells.

Fibers 2a, 2b and columnar bodies 5 are more preferably made of amorphous silicon dioxide, which allows the fibers to be flexible so as to intertwine more easily with each other.

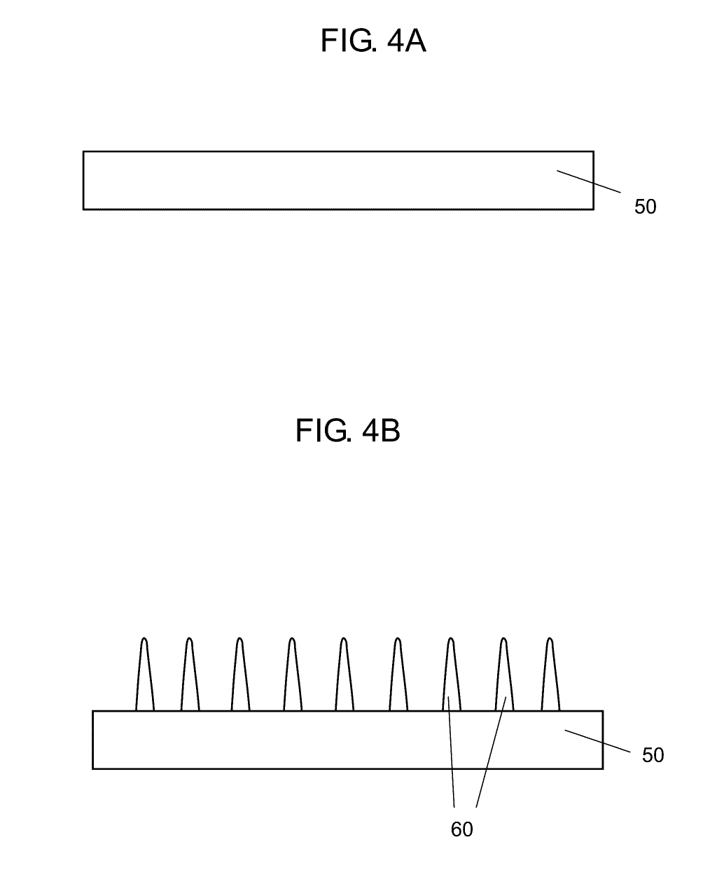

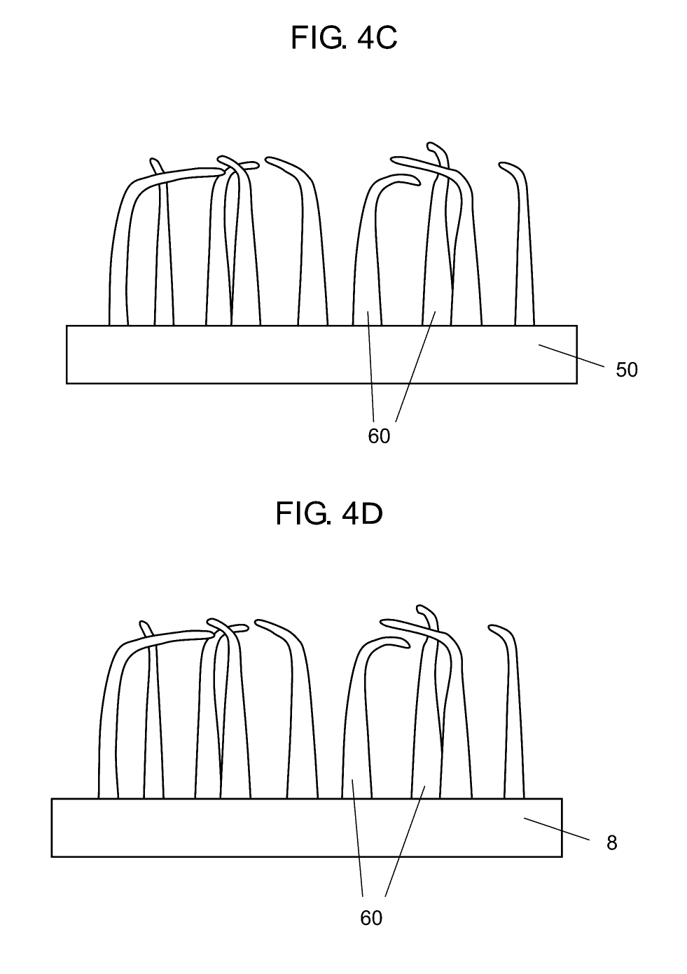

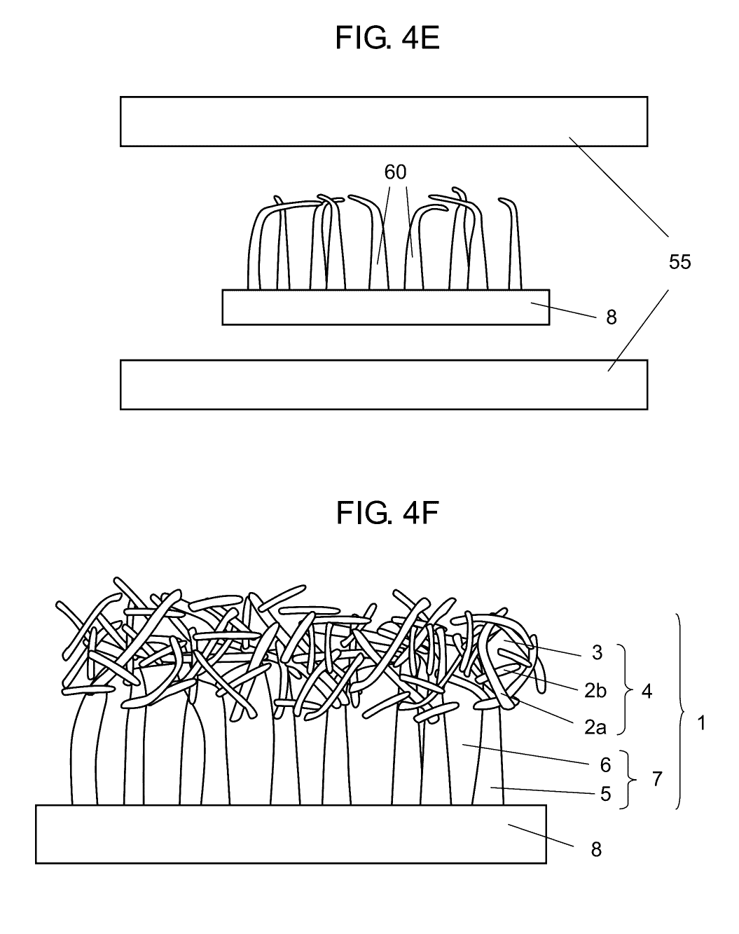

A method of manufacturing fibrous structure 1 will now be described as follows. FIGS. 4A to 4F are side views showing different steps of the method of manufacturing fibrous structure 1 in accordance with the first exemplary embodiment.

First, as shown in FIG. 4A, fiber-forming substrate 50 is prepared, which is coated with silicon (Si) particles. Alternatively, a silicon substrate may be used as substrate 50.

Next, substrate 50 is heated to a sintering temperature of 1000° C. to 1500° C. by a heater or other means. Substrate 50 is preferably heated in a deoxidized atmosphere, and may be heated in a vacuum. This is because introducing oxygen after the sintering temperature is reached can stabilize the sintering conditions, and hence, the fibers.

Substrate 50 heated to the sintering temperature is sintered at the constant temperature in an oxygen-containing gas atmosphere. Upon reaching the vapor pressure temperature, the silicon evaporates and then bonds to the oxygen contained in the gas, thereby becoming silicon monoxide (SiO). The silicon monoxide agglomerates and takes oxygen from the atmosphere, thereby becoming silicon dioxide (SiO₂). As a result, fibers 60 are deposited as shown in FIG. 4B.

The pressure during sintering can be made lower than the atmospheric pressure in order to lower the vapor pressure temperature of the silicon. This facilitates the evaporation of the silicon, thereby manufacturing more fibers. For example, substrate 50 can be sintered at a pressure of 10⁻² Pa to several thousands Pa to improve the fiber productivity.

When having nuclei, fibers 60 are efficiently deposited because the silicon monoxide agglomerates easily. It is, therefore, preferable to use a metal, such as Pt, Fe, Co, Ni, or Au as the nuclei; however, any other metal can be used, and it is not essential to have nuclei.

Fibers 60 can have different shapes depending on the temperature conditions under which fibers 60 are deposited. The shape of fibers 60 changes the conditions of columnar structure 7. Consequently, the temperature conditions change the ratio of sheet-like structure 4 to columnar structure 7; structure 4 is dominant in some cases, and structure 7 in the other cases.

FIG. 4C shows grown fibers 60. They are oriented substantially perpendicular to substrate 50 in regions near substrate 50 but are slightly bent in regions away from it.

In the regions near substrate 50, the silicon is more easily fed and bonds to oxygen to become silicon monoxide. Therefore, fibers 60 made of silicon dioxide are deposited more easily in the regions near substrate 50 than in those away from it, and consequently become thicker in the vicinity of substrate 50 than in the vicinity of their free ends, which are not fixed to substrate 50. Alternatively, fibers 60 can be more likely to bond to each other via deposited silicon dioxide and to become a bundle in the vicinity of substrate 50 and consequently become thicker than in the vicinity of their free ends.

To produce the fibers, it is possible to use an oxidizing (oxygen-supplying) gas, such as dinitrogen oxide (N₂O) or carbon monoxide (CO) instead of oxygen. These gases, however, contain impurities that oxygen does not contain, possibly affecting the growth of the fibers. Therefore, they need to be used under controlled concentration, temperature, and pressure.

Next, fibers 60 are separated from substrate 50 by applying stress as follows or may alternatively be separated by other methods described below.

In the case of using substrate 50 made of silicon, fibers 60 can be removed from substrate 50 by stress generated at an appropriate temperature due to the difference in thermal expansion coefficient between the silicon and the silicon dioxide.

Another method is to cut fibers 60 and substrate 50 apart with a diamond knife.

Still another method is as follows. Substrate 50 is soaked in pure water and the water is boiled to generate vapor explosion in a micro gap existing between fibers 60 and substrate 50. This vapor explosion cuts the bond between them.

Next, fibers 60 removed from substrate 50 are bonded to supporting substrate 8 as shown in FIG. 4D. They are bonded together by an adhesive made, for example, of a thermosetting or UV curable resin, but may alternatively be bonded by plasma treatment or any other method. Substrate 50 does not necessarily have to be removed and may be used as supporting substrate 8.

Substrate 8 can be made of glass, silicon, quartz, ceramic, metal, resin, etc. Specific examples of the resin include cyclic olefin copolymer (COC), cyclo-olefin polymer (COP), polycarbonate (PC), and polymethyl methacrylate (PMMA). Substrate 8 can alternatively be made of other materials, and it is not essential to use substrate 8.

If substrate 8 is made of a material with a higher melting point than silicon dioxide, such as silicon, quartz, or ceramic, fibers 60 made of silicon dioxide can be heated to partially melt and bonded to substrate 8. If, on the other hand, substrate 8 is made of a material with a lower melting point than silicon dioxide, such as glass or resin, substrate 8 can be heated to partially melt and bonded to fibers 60.

Alternatively, substrate 8 may have a layer with a lower melting point than silicon dioxide. This allows fibers 60 made of silicon dioxide to be bonded to substrate 8 without the need to melt them with heat. For example, a phosphorus silicate glass (PSG) film or a borosilicate glass (BSG) film is previously formed as an adhesive layer on substrate 8 made of silicon, quartz, or the like. When heated to 1000° C., fibers 60 made of silicon dioxide can be bonded to substrate 8 without the need to melt them. As a result, fibers 60 with a large surface area and high porosity can be bonded intact to substrate 8.

Further alternatively, substrate 8 may be previously coated with a polydimethylsiloxane (PDMS) film, so that fibers 60 can be affixed to substrate 8 without the need to melt them with heat. Thus, the use of the PDMS film facilitates the bonding between substrate 8 and fibers 60, thereby reducing the cost.

Next, as shown in FIG. 4E, fibers 60 formed on substrate 8 are compressed by press machine 55. The pressure is preferably not less than 1 MPa and not more than 20 MPa, in which fibers 60 are not broken into pieces and instead can be deformed at their free ends not fixed to substrate 8.

As shown in FIG. 4F, some of the free ends of fibers 60, which are not fixed to substrate 8, are broken and others are cut off by compression. Consequently, fibers 60 overlap or intertwine with each other to form sheet-like structure 4. On the other hand, in the vicinity of substrate 8, fibers 60 are oriented substantially perpendicular to sheet-like structure 4 and have a large diameter and a high stiffness. Thus, the regions of fibers 60 in the vicinity of substrate 8 maintain their structure even after compression. As a result, fibers 60 become columnar bodies 5 in the regions near substrate 8, and become sheet-like structure 4 in the regions away from substrate 8, thereby forming fibrous structure 1.

The silicon dioxide is deposited differently depending on the conditions, such as the pressure, oxygen concentration, and temperature in the atmosphere. Furthermore, the shape and density of fibers 60 change depending on the pressure during compression. Therefore, fibrous structure 1 can be formed in a desired shape by changing these conditions.

Fibers 60 may be compressed before fibrous structure 1 is fixed to substrate 8, but preferably be compressed after the fixation because it can prevent breakage of sheet-like structure 4 and columnar structure 7 during compression.

A side of sheet-like structure 4 can be flattened by bringing fibers 60 into contact with a highly flat substrate and compressing them using press machine 55 or other means.

As described above, sheet-like structure 4 has a high density and a large surface area because fibers 2b become embedded in first pores 3 formed by the intertwining of fibers 2a. Sheet-like structure 4 and columnar structure 7 are bonded to each other. Second pores 6 of columnar structure 7 are larger in size than first pores 3 of sheet-like structure 4. As a result, fibrous structure 1 has a high-density fiber configuration and a high (liquid) permeability.

Sheet-like structure 4 is hardly deformed under load because of its high density. Columnar structure 7, on the other hand, is hardly deformed under load in the direction in which columnar bodies 5 are oriented substantially perpendicular to sheet-like structure 4. Therefore, fibrous structure 1 is also hardly deformed under load in that direction. Note that fibers 2a and 2b used in the present embodiment are different in length from each other; alternatively, however, it is possible to use fibers different in thickness or aspect ratio.

### Second Exemplary Embodiment

Biochip 200 including fibrous structure 1 will now be described with reference to drawings. In the second exemplary embodiment, like components are labeled with like reference numerals with respect to the first exemplary embodiment, and hence the description thereof will be omitted.

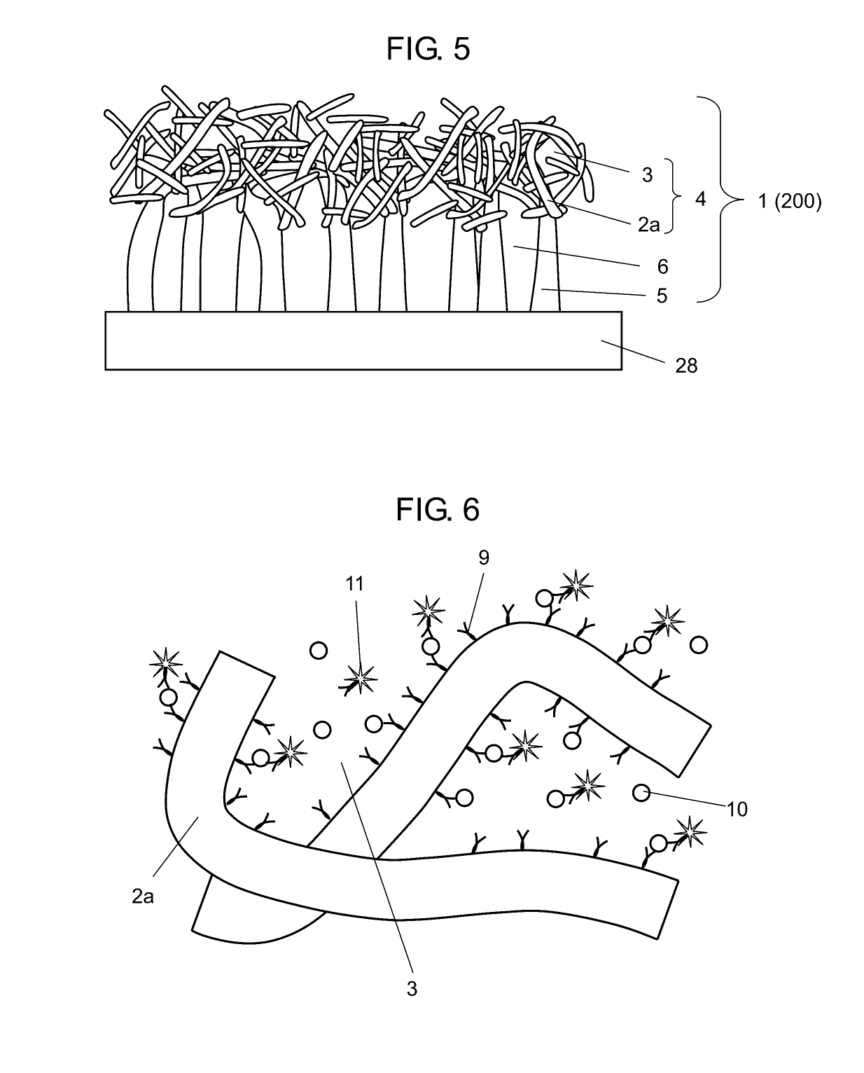

FIG. 5 is a conceptual view of biochip 200 including fibrous structure 1, which is fixed to supporting substrate 28. FIG. 6 is an enlarged view of an essential part of FIG. 5 and shows an example of a reaction in biochip 200. Biochip 200 includes fibrous structure 1, which includes a reaction field to which probes 9 are fixed.

Probes 9 are preferably fixed to the surfaces of fibers 2a by either physical adsorption or covalent bonding of spacer molecules (not shown). An aqueous solution containing detection targets 10 and labels 11 is reacted in first pores 3 of sheet-like structure 4, allowing labels 11 to be caught by probes 9 via targets 10.

Labels 11 that have not been caught are washed off. The amount of caught labels 11, which corresponds to the amount of targets 10, is measured to determine the amount of targets 10.

Labels 11 can be fluorescent molecules, such as Cy3 or Cy5. These molecules are exposed to light of the respective excitation wavelengths so as to detect fluorescence.

First pores 3 preferably have a maximum length of at least 0.05 μm to facilitate the reaction and bonding of probes 9, detection targets 10, and labels 11.

Supporting substrate 28 can be made of glass, silicon, quartz, ceramic, metal, resin, etc. Specific examples of the resin include cyclic olefin copolymer (COC), cyclo-olefin polymer (COP), polycarbonate (PC), and polymethyl methacrylate (PMMA). Substrate 28 is preferably made of a material with low autofluorescence and easiness of processing, but may alternatively be made of other materials. Fibrous structure 1 does not necessarily have to be fixed to substrate 28, but is preferably fixed to secure a given strength.

Sheet-like structure 4 has a high fiber density and first pores 3 because it is formed of fibers 2a overlapping or intertwining with each other. In other words, structure 4 includes a space (reaction field) in which probes 9, detection targets 10, and labels 11 can react easily. Therefore, when fibrous structure 1 is used in biochip 200, the reaction field has a large surface area within the focal length of a laser scanner or a fluorescence microscope used to detect fluorescence. In this case, the reaction field has a sufficient amount of probes 9 in the area effective for fluorescence detection, thereby having a high intensity of interaction, and hence, a high detection sensitivity of targets 10. In addition, sheet-like structure 4 is hardly deformed under a perpendicular force because of its high density.

Biochip 200 is washed more than two times with a cleaning solution. In the present exemplary embodiment, second pores 6 of columnar bodies 5 are larger than first pores 3 of sheet-like structure 4. This configuration allows the cleaning solution to be easily eliminated from fibrous structure 1, thereby facilitating the removal of probes 9, targets 10, and labels 11 adsorbed outside the reaction field. As a result, the background noise can be reduced.

Furthermore, columnar bodies 5 are thicker than fibers 2a and oriented substantially perpendicular to sheet-like structure 4 so as to be hardly deformed under load in that direction. In addition, since the above-mentioned side of sheet-like structure 4 is flattened by compression, the film thickness of fibrous structure 1 hardly changes after washing. The small variation in the film thickness of fibrous structure 1 reduces the variation in the number of targets 10, which is caused by the difference in focal length when a laser scanner or a fluorescence microscope is used for fluorescence detection.

In the present exemplary embodiment, fibers 2a and columnar bodies 5 are made of silicon dioxide. Since silicon dioxide has a low autofluorescence, when fluorescence is used for detection, the biochip has low background noise and high detection sensitivity. Moreover, silicon dioxide is superior to organic polymers in chemical and heat resistance, thereby facilitating the coating or adsorption of probes 9.

### Third Exemplary Embodiment

Cell culture substrate 300 including fibrous structure 1 will now be described with reference to drawings. In the third exemplary embodiment, like components are labeled with like reference numerals with respect to the first exemplary embodiment, and hence the description thereof will be omitted.

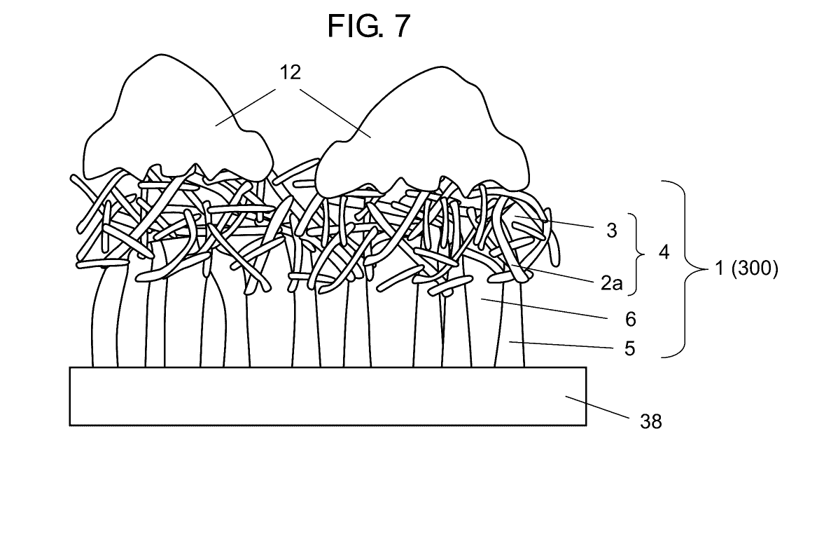

FIG. 7 is a conceptual view of cell culture substrate 300 including fibrous structure 1. Substrate 300 includes fibrous structure 1, which includes a region to which cells 12 are fixed.

Cells 12 are placed on top of fibers 2a in fibrous structure 1 so as to be adsorbed and cultivated there. Cells 12 are preferably adhesive cells.

FIG. 7 shows an example in which fibrous structure 1 is fixed to supporting substrate 38. Specific examples of the material for substrate 38 include glass, silicon, quartz, ceramic, metal, and resin. Specific examples of the resin include cyclic olefin copolymer (COC), cyclo-olefin polymer (COP), polycarbonate (PC), and polymethyl methacrylate (PMMA). Substrate 38 is preferably made of a highly transparent material to facilitate observation under an optical microscope, but may be made of other materials. Also, it is not always necessary that fibrous structure 1 be fixed to substrate 38.

In fibrous structure 1, second pores 6 formed among columnar bodies 5 are larger than first pores 3 formed between fibers 2a. This makes it easier to replace the culture medium and to supply nourishment than in the case that fibrous structure 1 is formed of sheet-like structure 4 alone. In addition, waste products are carried to the bottom of fibrous structure 1 through second pores 6 and easily discharged outside, without accumulating around cells 12 that are being cultivated. As a result, cells 12 can be cultivated for a longer period and have a higher survival rate.

As mentioned above, it is not always necessary that fibrous structure 1 be fixed to substrate 38. When fibrous structure 1 is not fixed to substrate 38, the culture medium flows more easily, making it easier to replace the medium and to supply nourishment, and also to discharge waste products. As a result, cells 12 can be cultivated for a longer period and have a higher survival rate.

Sheet-like structure 4 has a high fiber density because it is formed of fibers 2a overlapping or intertwining with each other. This configuration provides a large contact area between cells 12 and fibers 2a, thereby providing a high adhesive strength between cells 12 and substrate 300. As a result, cells 12 can be cultivated without being removed from substrate 300, thereby having a high survival rate. In addition, sheet-like structure 4 is hardly deformed under a perpendicular force because of its high density.

Furthermore, columnar bodies 5 are thicker than fibers 2a and oriented substantially perpendicular to sheet-like structure 4 so as to be hardly deformed under load in that direction. In addition, since the above-mentioned side of sheet-like structure 4 is flattened by compression, the variation in film thickness is small between different fibrous structures 1. Therefore, during the observation of cells 12 under a microscope, the variation in focal length is small between different regions of different fibrous structures 1, thereby facilitating the observation.

Fibers 2a and columnar bodies 5 are preferably made of silicon dioxide or other inorganic materials because silicon dioxide is resistant to heat and chemicals. Fibrous structure 1 made of silicon dioxide is resistant to temperatures of 1000° C. or more and can be easily surface-treated with heat. Fibrous structure 1 is also resistant to chemicals because silicon dioxide is not readily soluble in alkaline solutions. Thus, cell culture substrate 300 has high porosity per unit area and high resistance to heat and chemicals.

### Fourth Exemplary Embodiment

Filter 400 including fibrous structure 1 will now be described with reference to drawings. In the fourth exemplary embodiment, like components are labeled with like reference numerals with respect to the first exemplary embodiment, and hence the description thereof will be omitted.

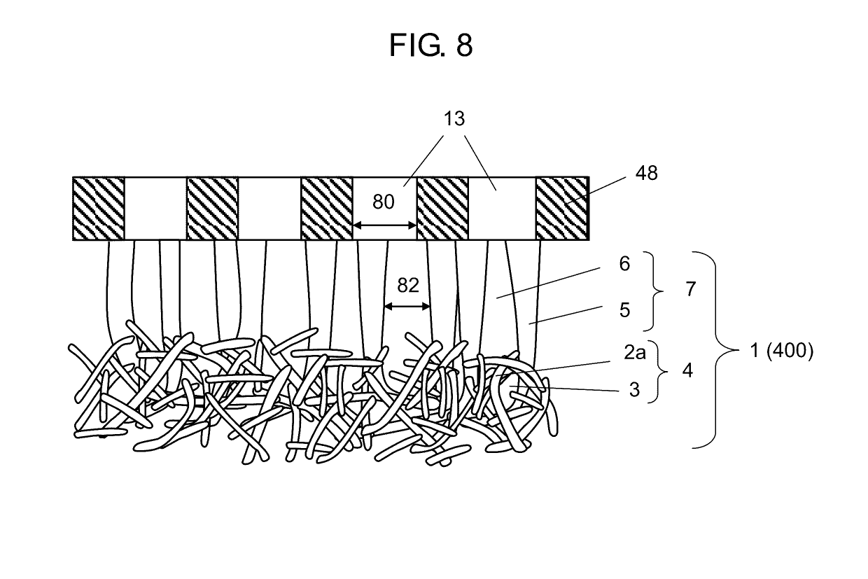

FIG. 8 is a conceptual view of filter 400 including fibrous structure 1. Filter 400 includes fibrous structure 1 in which either one of sheet-like structure 4 and columnar structure 7 functions as a sample inlet and the other as a sample outlet.

As shown in FIG. 8, supporting substrate 48 has through-holes 13. Through-holes 13 have a maximum diameter 80, which is larger than the maximum distance 82 between any adjacent ones of columnar bodies 5.

A sample containing various substances is introduced through the first side of substrate 48 (opposite to the side thereof to be bonded to columnar bodies 5). A filtered sample is collected through the first side of sheet-like structure 4 (opposite to the side thereof to be bonded to columnar bodies 5). As a result, large-sized substances contained in the sample are filtered out, and only specific small-sized substances are obtained.

Alternatively, it is possible to introduce a sample containing various substances through the first side of sheet-like structure 4 and to collect a filtered sample through the first side of substrate 48.

Since second pores 6 of columnar structure 7 are larger than first pores 3 of sheet-like structure 4, large-sized substances are caught in columnar structure 7 first, and then small-sized substances are caught in sheet-like structure 4 in a stepwise manner. Filter 400 is less clogged and allows the passage of a larger amount of sample than filters with uniform-sized pores.

Furthermore, through-holes 13 are larger than second pores 6 of columnar structure 7, so that the sample can be filtered in a three-step manner.

Columnar bodies 5, which are thicker than fibers 2a and oriented substantially perpendicular to substrate 48, are hardly deformed under load in that direction. Thus, the flow of the solution is not easily changed by external forces, so that the shapes of first and second pores 3 and 6 can be maintained during filtering. As a result, stable filtering can be performed regardless of the flow rate of the solution.

When fibrous structure 1 is made of silicon dioxide, fibrous structure 1 can be coated with various types of reagents because of its excellent chemical resistance. Furthermore, fibrous structure 1 is never melted or broken during high-temperature treatment because silicon dioxide is highly heat resistant. Thus, silicon dioxide is superior to organic polymers in chemical and heat resistance, so that filter 400 can be easily subjected to surface treatment for improving adsorption of samples.

Silicon dioxide also has a larger Young's modulus (elastic coefficient) than polymers. The Young's modulus is a physical property indicating difficulty of change. The Young's modulus of silicon dioxide is 30 to 75 GPa, whereas those of polymers are 0.01 to 5 GPa. Therefore, fibrous structure 1 made of silicon dioxide is not susceptible to deformation under external forces, such as the flow of the solution, and maintains its pore structure during filtering. As a result, stable filtering can be performed regardless of the flow rate of the solution.

Substrate 48 can be made of glass, silicon, quartz, ceramic, metal, resin, etc. Specific examples of the resin include cyclic olefin copolymer (COC), cyclo-olefin polymer (COP), polycarbonate (PC), and polymethyl methacrylate (PMMA). Substrate 48 can alternatively be made of other materials, and fibrous structure 1 does not necessarily have to be fixed to substrate 48.

As described above, fibrous structure 1 in accordance with the exemplary embodiments has a high density because fibers 2a overlap or intertwine with each other. In addition, columnar bodies 5, which are in contact with sheet-like structure 4, can maintain their strength in the oriented direction and hold second pores 6 between themselves.

In biochip 200 including fibrous structure 1, sheet-like structure 4 functions as the reaction field, allowing biochip 200 to have a large surface area and a large amount of adsorbed probes 9. This provides a strong interaction between the targets and probes 9 so that targets 10 can be detected at high sensitivity. In addition, second pores 6 facilitate the discharge of the solution, so as to reduce nonspecific adsorption, and hence, background noise.

Cell culture substrate 300 including fibrous structure 1 has a large area where fibrous structure 1 and cells 12 are bonded to each other, so that cells 12 and substrate 300 can be bonded together with high adhesive strength. In addition, the pores formed between columnar bodies 5 facilitate the discharge of waste products, thereby providing a high (liquid) permeability.

Filter 400 including fibrous structure 1 filters out large-sized substances first and then small-sized ones in a stepwise manner so as to cause less clogging, allowing the passage of a larger amount of sample than other filters with uniform-sized pores. Fibrous structure 1 also performs stable filtering because it is not susceptible to deformation and maintains its pore structure during filtering regardless of the flow rate of the solution.

As described in these exemplary embodiments, fibrous structure 1 has a high density and a high (liquid) permeability.

### INDUSTRIAL APPLICABILITY

The fibrous structure of these exemplary embodiments is useful in biochips, cell culture substrates, and filters.

### REFERENCE MARKS IN THE DRAWINGS

fibrous structure

2a, 2b fiber

3 first pore

4 sheet-like structure

5 columnar body

6 second pore

7 columnar structure

8, 28, 38, 48 supporting substrate

9 probe

10 detection target

11 label

12 cell

13 through-hole

50 fiber-forming substrate

55 press machine

60 fiber

70 first direction

80 diameter

82 distance

200 biochip

300 cell culture substrate

400 filter

1. A fibrous structure comprising:
a sheet-like structure including:
a plurality of first fibers intertwining with each other; and
first pores formed among the first fibers, and
a columnar structure including:
a plurality of columnar bodies each made of a second fiber and oriented in a first direction; and
second pores formed between the columnar bodies,
wherein the columnar bodies are each in contact, at a first end thereof, with the sheet-like structure.

2. The fibrous structure of claim 1, wherein the columnar bodies have a larger average thickness than the first fibers.

3. The fibrous structure of claim 1, wherein the second pores have a larger volume than the first pores.

4. The fibrous structure of claim 1, further comprising a supporting substrate connected to a second end opposite to the first end of each of the columnar bodies.

5. The fibrous structure of claim 4, wherein the supporting substrate is made of at least one of glass, silicon, quartz, ceramic, resin, and metal.

6. The fibrous structure of claim 1, wherein the first and second fibers are made of silicon dioxide.

7. The fibrous structure of claim 1, wherein the first and second fibers are made of amorphous silicon dioxide.

8. The fibrous structure of claim 1, wherein the first and second fibers are made of an identical material.

9. A biochip comprising the fibrous structure of claim 1 including a reaction field in which probes are fixed.

10. A cell culture substrate comprising the fibrous structure of claim 1 including a region in which cells -are bonded.

11. A filter comprising the fibrous structure of claim 1, wherein one of the sheet-like structure and the columnar structure functions as a sample inlet and an other functions as a sample outlet.

12. The filter of claim 11, further comprising a supporting substrate having through-holes and connected to a second end opposite to the first end of each of the columnar bodies.

13. The filter of claim 12, wherein the through-holes have a maximum diameter larger than a maximum distance between any adjacent ones of the columnar bodies.
